(19) United States
(12) Patent Application Publication
(10) Pub. No.: US 2011/0245683 A1
(43) Pub. Date: Oct. 6, 2011

(54) OPTICAL IMAGING DIAGNOSTIC APPARATUS AND THE DISPLAY CONTROL METHOD THEREOF

(75) Inventor: Yuuji ONIMURA, Fujinomiya-shi (JP)

(73) Assignee: TERUMO KABUSHIKI KAISHA, Shibuya-ku (JP)

(21) Appl. No.: 13/075,900

(22) Filed: Mar. 30, 2011

(30) Foreign Application Priority Data
Mar. 30, 2010 (JP) 2010-078608

(51) Int. Cl. A61B 6/00

(52) U.S. Cl. 600/476

(57) ABSTRACT

An optical imaging diagnostic apparatus includes a calculation unit for respectively calculating signal-intensity change in the circumferential direction of the circumference of the transmitting and receiving unit and signal-intensity change in the radial direction of a body lumen respectively with respect to a plurality of line data used for generation of a cross-sectional image displayed; a specifying unit for judging, based on the calculated result in the calculation unit, whether or not there exists scatter which scatters the light transmitted from the transmitting and receiving unit in an blood flow area of the body lumen and for specifying the position on the displayed cross-sectional image of the scatter in case of being judged that there exists the scatter; and a display unit for displaying a marker indicating that the scatter exists at the specified position on the displayed cross-sectional image.

This application contains subject matter disclosed in, and claims priority to, Japanese Patent Application No. 2010-078608 filed in the Japanese Patent Office on Mar. 30, 2010, the entire content of which is incorporated herein by reference.

### TECHNOLOGICAL FIELD

The present invention generally relates to an optical imaging diagnostic apparatus and a display control method.

### BACKGROUND DISCUSSION

In the past, there has been used an optical coherent tomography (OCT) apparatus, an example of which is disclosed in Japanese Unexamined Patent Publication No. 2001-79007, and an optical frequency domain imaging (OFDI) apparatus utilizing wavelength sweep, which is an improvement type of apparatus for diagnosing arterioscleosis, for a diagnosis before operation at the time of treatment inside a blood vessel depending on a high functional catheter such as a balloon catheter, a stent and the like, or for a result confirmation after operation Hereinafter, in the present specification, the optical coherent tomography (OCT) apparatus and the optical frequency domain imaging (OFDI) apparatus utilizing wavelength sweep are generically referred to as “optical imaging diagnostic apparatus”.

In the optical imaging diagnostic apparatus, an optical probe unit inserted with an imaging core which is attached with an optical lens and an optical mirror (transmitting and receiving unit) at a distal end of an optical fiber is inserted into a blood vessel, and a radial scan in a blood vessel is carried out by emitting a measurement light into the blood vessel from the transmitting and receiving unit at the distal end while rotating the imaging core and concurrently, by receiving a reflected light from a biological tissue. Then, a coherent light is produced by making the light-received reflected light and a reference light interfere with each other, and a cross-sectional image of the blood vessel is derived based on the coherent light.

Generally, when carrying out the development or creation of a cross-sectional image by using such an optical imaging diagnostic apparatus, it is necessary to preliminarily carry out flushing, occlusion or the like to remove the blood inside the blood vessel for which the measurement light is emitted. One reason is because blood cells are fairly strong scatterers and therefore, when there exist blood cells in the blood vessel, the measurement light is attenuated considerably and so it is difficult to produce a desired cross-sectional image.

The word “flushing” refers to a physiological salt solution, lactate ringer, a contrast agent or the like that is discharged in the imaging target area so the blood inside the blood vessel is preliminarily removed. The term “occlusion” refers to the blood vessel being blocked temporarily by a balloon or the like.

Even in a case in which flushing, occlusion or the like is carried out on one hand, it is not always true that the blood in the blood flow area inside the blood vessel can be removed perfectly. Even in such a case, if the blood quantity remaining in the blood vessel is a predetermined quantity or more, it is possible for a user to confirm this by the developed cross-sectional image, but in a case in which the quantity of the bloods remained in the blood vessel is rather fine (minute), or in such a case in which floating thrombi remain, it is difficult to confirm the existence of small amounts of blood or thrombi in the developed cross-sectional image.

However, blood is a strong scatterer as mentioned above, so that even in a case in which a relatively minute or small amount of blood or thrombi exist, the measurement light is attenuated in the backward area of the direction to which the measurement light is emitted. Such a phenomenon is similar to a phenomenon of attenuation of the measurement light in the backward area which is caused by macrophage accumulated on the blood-vessel surface. These two phenomena are difficult for a user to distinguish at the time of diagnosis.

Consequently, it is difficult to confirm the existence on the cross-sectional image which is developed or generated. Also, with respect to the minute or fine blood and thrombi (hereinafter, these are referred to as fine scatter or fine scattering) which cause the attenuation of measurement light in the blood flow area inside the blood vessel, it is preferable for a user to be able to identify the presence or non-presence (absence) of such fine scatter on the cross-sectional image.

### SUMMARY

The optical imaging diagnostic apparatus and method disclosed here make it possible for the presence or absence of fine scatter to be identified on the cross-sectional image.

An optical imaging diagnostic apparatus has a probe including a transmitting and receiving unit for carrying out continuous transmitting of light which is reflected from biological tissue and received as obtained reflected light by the transmitting and receiving unit, with the transmitting and receiving unit being axially movable inside a body lumen while also rotating the transmitting and receiving unit, to generate a plurality of cross-sectional images in an axial direction of the biological tissue using line data of coherent light produced by interference between the obtained reflected light and reference light, with the cross-sectional images being displayed. The optical imaging diagnostic apparatus comprises: calculation means for respectively calculating signal-intensity change in a circumferential direction of a circumference of the transmitting and receiving unit and signal-intensity change in a radial direction of the body lumen with respect to a plurality of line data used for generating the displayed cross-sectional image; specifying means for judging, based on a result calculated by the calculation means, whether or not there exists scatter which scatters the light transmitted from the transmitting and receiving unit in a blood flow area inside the body lumen, and for specifying a position of the scatter on the displayed cross-sectional image when it is judged that the scatter exists; and a display which displays on the display a marker indicating that the scatter exists and indicating on the displayed cross-sectional image the position of the scatter specified by the specifying means

Another aspect involves display control method in an optical imaging diagnostic apparatus, which apparatus comprises a probe including a transmitting and receiving unit for carrying out continuous transmitting of light which is reflected from biological tissue and received as obtained reflected light by the transmitting and receiving unit, with the transmitting and receiving unit being axially movable inside a body lumen while also rotating, to generate a plurality of cross-sectional images in an axial direction of the biological tissue using line data of coherent light produced by interference between the obtained reflected light and reference light, with the cross-sectional images being displayed. The method comprises: calculating both signal-intensity change in a circumferential direction of a circumference of the transmitting and receiving unit and signal-intensity change in a radial direction of the body lumen respectively with respect to plural line data used to generate the displayed cross-sectional image; judging, based on both the signal-intensity change in the circumferential direction of the circumference of the transmitting and receiving unit and the signal-intensity change in the radial direction of the body lumen, whether or not scatter exists which scatters the light transmitted from the transmitting and receiving unit in a blood flow area of the body lumen; and specifying, when it is determined that scatter exist, a position of the scatter on the displayed cross-sectional image. The specifying of the position of the scatter on the displayed cross-sectional image includes displaying a marker on the displayed cross-sectional image which identifies the position of the scatter.

According to the apparatus and method disclosed here, it becomes possible in an optical imaging diagnostic apparatus to identify the presence or non-presence of a fine scatter on a displayed cross-sectional image.

### BRIEF DESCRIPTION OF THE DRAWINGS

FIG. 1 is a perspective view of an example of an imaging diagnostic apparatus disclosed here.

FIG. 2 is a block diagram of an example of an optical coherent tomography apparatus.

FIG. 3 is a block diagram of an example of an optical frequency domain imaging apparatus utilizing wavelength sweep.

FIG. 4 is cross-sectional perspective view illustrating an imaging core distal end inserted into an optical probe unit of an optical imaging diagnostic apparatus and illustrating an operation of an imaging core in a state of being inserted into a blood vessel.

FIGS. 5A and 5B are cross-sectional perspective views illustrating an imaging core operation at the time of measurement in an optical imaging diagnostic apparatus and a generation process of a blood vessel cross-sectional image.

FIG. 6 is a diagram explaining the features and other functional aspects of a signal processing unit in an optical imaging diagnostic apparatus.

FIG. 7A is a flowchart showing the fine scatter detection process disclosed here.

FIG. 7B is a diagram showing one example of a blood flow area on line data.

FIGS. 8A and 8B are diagrams showing one example of a user interface in a fine scatter detection process.

FIG. 9 is a diagram illustrating features of the signal processing unit in an optical imaging diagnostic apparatus.

FIG. 10 is a flowchart showing a fine scatter detection process.

FIG. 11 is a diagram showing one example of a user interface in a fine scatter detection process.

FIG. 12 is a flowchart showing a cross-sectional image correction process.

FIG. 13 is a flowchart showing a cross-sectional image correction process.

### DETAILED DESCRIPTION

The description which follows describes details and aspects of respective embodiments of an optical imaging diagnostic apparatus and a display control method disclosed here by way of example.

### First Embodiment

1. Overall Construction of Imaging Diagnostic Apparatus

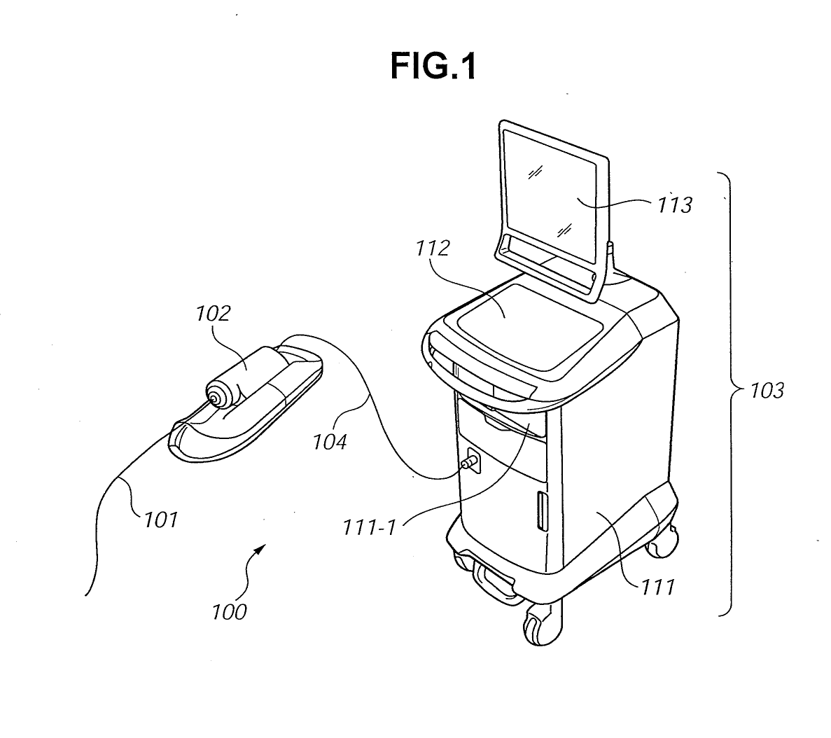

FIG. 1 illustrates, for the exterior, the construction and features of an optical imaging diagnostic apparatus (optical coherent tomography apparatus or optical frequency domain imaging apparatus utilizing wavelength sweep) 100 disclosed here by way of example as a first embodiment.

As shown in FIG. 1, the optical imaging diagnostic apparatus 100 is provided with an optical probe unit 101, a scanner & pull-back unit 102 and a steering control apparatus 103. The scanner & pull-back unit 102 and the steering control apparatus 103 are connected by a signal line 104.

The optical probe unit 101 is inserted directly inside a body lumen of a blood vessel or the like and transmits the transmitted measurement light continuously to a biological tissue and concurrently, includes an imaging core provided with a transmitting and receiving unit for receiving the reflected light from the biological tissue continuously. A state of the biological tissue is measured by using the imaging core. The scanner & pull-back unit 102 is constructed such that the optical probe unit 101 is attachable and detachable, and operates to effect a radial operation of the imaging core inserted in the optical probe unit 101 based on the riving operation of an installed motor.

The steering control apparatus 103 is configured so that, during operation when carrying out measurements, various kinds of setting values can be inputted. The steering control apparatus 103 also operates to process data obtained by the measurement and to display it as a blood vessel cross-sectional image.

In the steering control apparatus 103, a reference numeral 111 indicates a main body control unit which processes the data obtained by the measurement and outputs the processed result. A printer & DVD recorder 111-1 prints the processed result from the main body control unit 111 and stores such result as data signals.

An operation panel 112 is provided. A user is able to carry out inputs of various kinds of setting values and instruction through the operation panel 112. An LCD monitor 113 is a display apparatus that displays the processed result produced in the main body control unit 111.

2. Features and Construction of Optical Coherent Tomography Apparatus

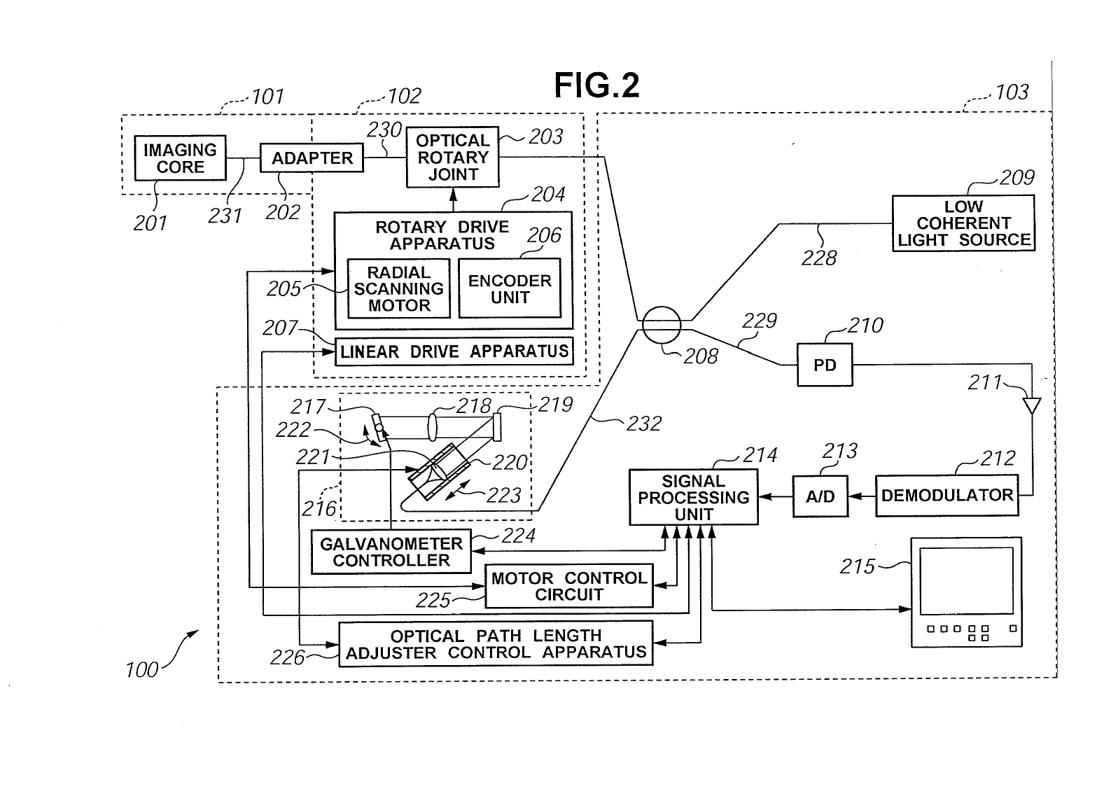

FIG. 2 illustrates aspects of the optical coherent tomography apparatus within the optical imaging diagnostic apparatus 100 relating to this embodiment disclosed by way of example.

The optical coherent tomography apparatus includes a low coherent light source 209 of a super high intensity light-emitting diode or the like. The low coherent light source 209 outputs a low coherent light whose wavelength is around 1310 nm and which shows coherence only in such a short distance range in which a coherent-able distance thereof (coherent length) is around a few μm to ten and a few μm.

Consequently, when light is divided into two light paths and thereafter the light paths are mixed again, the light is detected as a coherent light in a case in which the difference between the two optical path lengths from the point at which the light is divided to a point at which they are mixed is within a short distance range of around a few μm to ten and a few μm, and the light is determined not to be coherent light in a case in which the difference of the optical path lengths is larger than that.

The light of the low coherent light source 209 enters into one end of a first single mode fiber 228 and is transmitted to the distal end surface side. The first single mode fiber 228 is connected optically with a second single mode fiber 229 and a third single mode fiber 232 by a photo coupler unit 208.

The photo coupler unit is an optical component which makes it possible to divide one optical signal into two or more outputs and/or to combine two or more inputted optical signals for one output. Here, the light of the low coherent light source 209 is transmitted and divided into three optical paths at the maximum by the photo coupler unit 208.

The scanner & pull-back unit 102 is provided on the distal end side ahead of the photo coupler unit 208 of the first single mode fiber 228. In the inside of a rotary drive apparatus 204 of the scanner & pull-back unit 102, there is provided an optical rotary joint 203 for connecting between a non-rotary portion (fixed portion) and a rotary portion (rotary drive portion) and for transmitting the light.

Further, the distal end side of a fourth single mode fiber 230 on the inside of the optical rotary joint 203 is connected in a freely detachable manner with a fifth single mode fiber 231 of the optical probe unit 101 through an adapter 202. Thus, the light from the low coherent light source 209 is transmitted to the fifth single mode fiber 231 which is passed-through in the imaging core 201 provided with the transmitting and receiving unit, which repeats the transmitting and receiving of the light and which is rotary-drivable.

The light transmitted to the fifth single mode fiber 231 is emitted while radially operating with respect to the biological tissue inside the blood vessel from the transmitting and receiving unit arranged at the distal end of the imaging core 201. Then, a portion of the reflected light scattered on the surface or on the inside of the biological tissue is taken-in or received by the imaging core 201 and returns to the first single mode fiber 228 side through a reverse optical path, and a portion thereof moves to the second single mode fiber 229 side by the photo coupler unit 208. Then, it is light-received by a photo detector (for example, photo diode 210) owing to a fact that it is emitted from one end of the second single mode fiber 229.

The rotary drive portion side of the optical rotary joint 203 is rotatingly driven by a radial scanning motor 205 of the rotary drive apparatus 204. A rotary angle of the radial scanning motor 205 is detected by an encoder unit 206. Further, the scanner & pull-back unit 102 is provided with a linear drive apparatus 207 and movement (axial-direction operation) in an axial direction (distal direction of the body lumen and opposite direction thereof) of the imaging core 201 is defined based on an instruction from a signal processing unit 214. The axial-direction operation occurs by virtue of the fact that the linear drive apparatus 207 makes the scanner including the optical rotary joint 203 move based on a control signal from the signal processing unit 214.

At that time, only the imaging core 201 inserted into a catheter sheath moves axially while the catheter sheath of the optical probe unit 101 is maintained in a fixed position in the blood vessel so that it is possible to carry out the axial-direction operation without injuring a blood vessel wall.

On the other hand, a variable mechanism 216 of the optical path length for changing the optical path length of the reference light is provided on the distal end side ahead the photo coupler unit 208 of the third single mode fiber 232 (on the reference light path).

The variable mechanism 216 of this optical path length is provided with a first optical path length changing unit for relatively speedily changing the optical path length which corresponds to an inspection region in the depth direction (direction of emission of the measurement light) of the biological tissue and a second optical path length changing unit for changing the optical path length which corresponds to fluctuation of the length thereof such that there can be absorbed the fluctuation of the length of the individual optical probe unit 101 in the case of the optical probe unit 101 being exchanged.

Further, in the variable mechanism 216, facing the distal end of the third single mode fiber 232, there is arranged, through a collimating lens 221 which is freely movable in the direction shown by the arrow 223, with a mirror 219 which is mounted on an one-axis stage 220 together with this distal end. Also, there is mounted, through a mirror 218 corresponding to this mirror 219 (diffraction lattice), with a galvanometer 217 which is rotatable by a fine angle as the first optical path length changing unit. This galvanometer 217 is rotated fairly speedily in the direction of the arrow 222 by a galvanometer controller 224.

The galvanometer 217 is a device which reflects light by a mirror of the galvanometer and it is constructed such that the mirror mounted on a movable portion thereof is to be relatively speedily rotated by applying an AC drive signal to the galvanometer which functions as a reference mirror.

That is to say, owing to a fact that the drive signal is applied with respect to the galvanometer 217 from the galvanometer controller 224 and it is relatively speedily rotated by the drive signal in the direction of the arrow 222, the optical path length of the reference light is changes fairly quickly only by the optical path length which corresponds to the inspection region in the depth direction of the biological tissue. One cycle of the change of this optical path difference becomes a cycle for obtaining coherent light for one line.

The one-axis stage 220 functions as the second optical path length changing unit having such an amount of variable range of optical path length, which can absorb fluctuations in the optical path length of the optical probe unit 101 in the event of an exchange of the optical probe unit 101. Further, the one-axis stage 220 is also provided with a function as an adjuster for adjusting an offset. For example, even in a case in which the distal end of the optical probe unit 101 is not closely-attached to the surface of the biological tissue, it is possible, by changing the optical path length by the one-axis stage 220, to set it in a state of interfering with the reflected light from the surface position of the biological tissue.

The light whose optical path length is changed by the variable mechanism 216 of the optical path length is mixed with the reflected light obtained from the first single mode fiber 228 side by the photo coupler unit 208 which is provided along the third single mode fiber 232 and is light-received as coherent light by the photo diode 210.

The coherent light which is light-received by the photo diode 210 in this manner is photoelectrically converted and amplified by an amplifier 211 and thereafter, is inputted to a demodulator 212.

In the demodulator 212, a demodulation process for extracting only the signal component of the coherent light is carried out and the output thereof is inputted to an A/D converter 213.

In the A/D converter 213, there is produced digital data as “coherent light data” of one line by sampling the coherent light signal, for example for 200 points. In this case, the sampling frequency becomes a value dividing one scanning time period of the optical path length by 200.

The coherent light data per line unit which is produced by the A/D converter 213 is inputted to a signal processing unit 214. In the signal processing unit 214, by carrying out a predetermined process with respect to the coherent light data, there is produced line data which are the coherent light intensity data in the depth direction of the biological tissue and thereafter, by converting the line data to the video signal, there is produced a cross-sectional image at each position in the axial direction inside the blood vessel, and it is outputted to a user interface apparatus 215 by a predetermined frame rate (corresponding to LCD monitor 113 and operation panel 112 in FIG. 1).

The signal processing unit 214 is connected further to an optical path length adjuster control apparatus 226. The signal processing unit 214 carries out the control of the position of the one-axis stage 220 through the optical path length adjuster control apparatus 226. Also, the signal processing unit 214 is connected to a motor control circuit 225 and controls the rotary drive of the radial scanning motor 205.

Further, the signal processing unit 214 is connected to the galvanometer controller 224 for controlling the scan of the optical path length of the reference mirror (galvanometer mirror) and the galvanometer controller 224 outputs the drive signal to the signal processing unit 214. In the motor control circuit 225, synchronization with the galvanometer controller 224 is taken by using this drive signal.

3. Construction of Optical Frequency Domain Imaging Apparatus Utilizing Wavelength Sweep

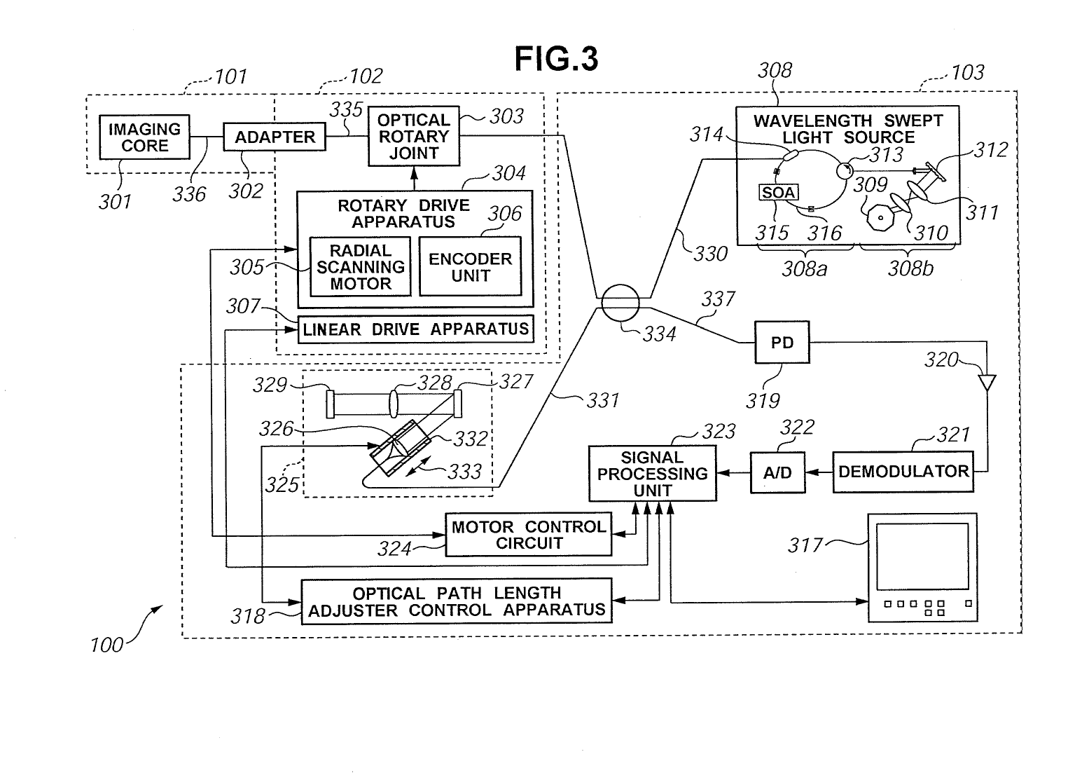

Referring to FIG. 3, set forth below is a description of the construction and functional aspects of an optical frequency domain imaging apparatus utilizing wavelength sweep within the optical imaging diagnostic apparatus 100 according to one embodiment disclosed by way of example.

Referring to FIG. 3, the optical frequency domain imaging apparatus 100 utilizing wavelength sweep includes a wavelength swept light source 308. In this embodiment, the wavelength swept light source 308 is a Swept-Laser. The wavelength swept light source 308 using the Swept-Laser is one kind of an Extended-Cavity-Laser which is composed of an optical fiber 316 connected with SOA315 (semiconductor optical amplifier) in a ring shape and a polygon scanning filter (308b).

The light outputted from the SOA315 proceeds inside the optical fiber 316 and enters in the polygon scanning filter 308b, and the wavelength selected here is amplified by the SOA315 and finally, it is outputted from a coupler 314.

In the polygon scanning filter 308b, the wavelength is selected depending on the combination of a diffraction lattice 312 for light-splitting the light and a polygon mirror 309. Specifically, the light which is light-split by the diffraction lattice 312 is focused on the surface of the polygon mirror 309 depending on two pieces of lenses (310, 311). Thus, only the light of the wavelength perpendicular to the polygon mirror 309 returns to the same optical path and is outputted from the polygon scanning filter 308b, so that it is possible to carry out the time sweep of the wavelength by rotating the polygon mirror 309.

For the polygon mirror 309, for example, an icosadodecahedron mirror is used and the rotation speed thereof is around 50000 rpm. Owing to the wavelength sweep system in which the polygon mirror 309 and the diffraction lattice 312 are combined, it becomes possible to employ the wavelength sweep of relatively high speed and relatively high power.

The light of the wavelength swept light source 308, which is outputted from a coupler 314, enters one end of a first single mode fiber 330 and is transmitted to the distal end side. The first single mode fiber 330 is optically connected with a second single mode fiber 337 and a third single mode fiber 331 in a photo coupler unit 334 on the way. Therefore, the light which enters the first single mode fiber 330 is transmitted by being split into three optical paths at the maximum by this photo coupler unit 334.

On the distal end side ahead of the photo coupler unit 334 of the first single mode fiber 330, there is provided, in a rotary drive apparatus 304, with an optical rotary joint 303 which connects a non-rotary portion (fixed portion) and a rotary portion (rotary drive portion) and which transmits the light.

Further, the distal end side of a fourth single mode fiber 335 on the inside of the optical rotary joint 303 is connected in a freely detachable manner through a fifth single mode fiber 336 and an adapter 302 of the optical probe unit 101. Consequently, the light from the wavelength swept light source 308 is transmitted to the fifth single mode fiber 336 which is passed-through an imaging core 301 and which is rotary-drivable.

The transmitted light is emitted from the transmitting and receiving unit arranged at a distal end of the imaging core 301 with respect to the biological tissue while being radially operated. Then, a portion of the reflected light which is scattered on the surface of or on the inside of the biological tissue is taken-in or received by the imaging core 301 and returns to the first single mode fiber 330 side through the reverse optical path. Further, the light is light-received by a photo detector (for example, photo diode 319) owing to a fact that a portion of the light moves to the second single mode fiber 337 side by the photo coupler unit 334 and is emitted from one end of the second single mode fiber 337.

The rotary drive portion side of the optical rotary joint 303 is rotatingly driven by a radial scanning motor 305 of the rotary drive apparatus 304. Also, rotary angle of the radial scanning motor 305 is detected by an encoder unit 306. Further, the scanner & pull-back unit 102 is provided with a linear drive apparatus 307 and defines the axial-direction operation of the imaging core 301 based on an instruction from a signal processing unit 323.

A variable mechanism 325 of the optical path length is provided for fine-adjusting the optical path length of the reference light at a distal end on the opposite side with respect to the photo coupler unit 334 of the third single mode fiber 331.

The variable mechanism 325 of this optical path length is provided with the optical path length changing unit for changing the optical path length which corresponds to the fluctuation of the length thereof such that fluctuation in the length of the individual optical probe unit 101 can be absorbed in case of the optical probe unit 101 being exchanged.

The third single mode fiber 331 and a collimating lens 326 are provided on a one-axis stage 332 which is freely movable in the optical axial direction as shown by an arrow 333, and they form the optical path length changing unit.

Specifically, the one-axis stage 332 functions as the optical path length changing unit having such an amount of variable range of optical path length, which can absorb the fluctuation of the optical path length of the optical probe unit 101 in the case of exchanging the optical probe unit 101. Further, the one-axis stage 332 also serves as an adjuster for adjusting an offset. For example, even in a case in which the distal end of the optical probe unit 101 is not closely-attached to the surface of the biological tissue, it becomes possible, by changing the optical path length by the one-axis stage, to set it in a state of interfering with the reflected light from the surface position of the biological tissue.

The light whose optical path length is fine-adjusted by the variable mechanism 325 of the optical path length is mixed with the reflected light obtained from the first single mode fiber 330 side by the photo coupler unit 334 which is provided on the way of the third single mode fiber 331 and it is light-received by the photo diode 319.

The coherent light which is light-received by the photo diode 319 in this manner is photoelectrically converted and amplified by an amplifier 320 and thereafter, is inputted to a demodulator 321. In the demodulator 321, a demodulation process for extracting only the signal component of the coherent light is carried out and the output thereof is inputted to an A/D converter 322 as the coherent light signal.

In the A/D converter 322, there is produced one line of digital data “coherent light data” by sampling the coherent light signal, for example, for 2048 points by 180 MHz. The sampling frequency is set to be 180 MHz based on the assumption that about 90% of the cycle (12.5 μsec) of the wavelength sweep is to be extracted as the digital data of 2048 points in case of setting the repeat frequency of the wavelength sweep to be 80 kHz. The apparatus and method here are not limited in this respect.

The coherent light data per line unit produced in the A/D converter 322 is inputted to the signal processing unit 323. In the signal processing unit 323, the coherent light data are frequency-decomposed depending on an FFT (Fast Fourier Transform) and then, there are generated line data which are the coherent light intensity data in the depth direction. By coordinate-converting that data, there is formed a cross-sectional image at each position in the axial direction of the inside of the blood vessel and it is outputted to a user interface apparatus 317 (which corresponds to LCD monitor 113 and operation panel 112 in FIG. 1) at a predetermined frame rate.

The signal processing unit 323 is connected further to an optical path length adjuster control apparatus 318. The signal processing unit 323 carries out the control of the position of the one-axis stage 332 through the optical path length adjuster control apparatus 318. Also, the signal processing unit 323 is connected to a motor control circuit 324 and receives a video synchronization signal of the motor control circuit 324. In the signal processing unit 323, the generation of the cross-sectional image is carried out in synchronization with the received video synchronization signal.

In addition, the video synchronization signal of this motor control circuit 324 is transmitted also to the rotary drive apparatus 304 and in the rotary drive apparatus 304, the drive signal in synchronization with the video synchronization signal is outputted.

4. Construction of Distal End of Imaging Core Inserted in Optical Probe Unit and Operation of the Imaging Core

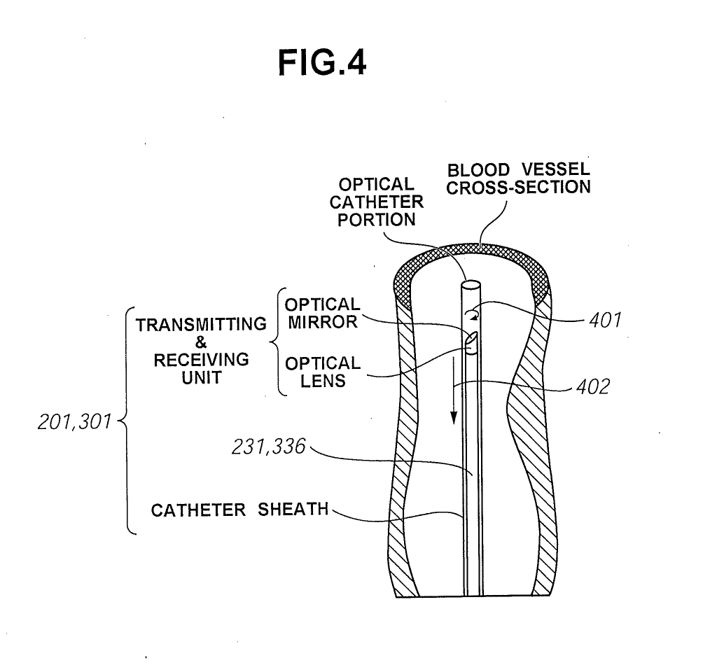

Set forth below with reference to FIG. 4 is a description of the construction of a distal end of the imaging core 201, 301 inserted in the optical probe unit 101 and the operation of the imaging core 201, 301. FIG. 4 illustrates the construction of a distal end of the imaging core 201, 301 inserted in the optical probe unit 101, with the optical probe unit 101 inserted into a blood vessel.

As shown in FIG. 4, the imaging core 201, 301 provided with the transmitting and receiving unit and the optical fiber 231, 336 is inserted in the optical probe unit 101, and it is constructed to operate linearly in the direction of the arrow 402 while rotating in the direction of the arrow 401 inside the optical probe unit 101 which is inserted into the blood vessel (it is constructed to carry out radial operation).

The transmitting and receiving unit is provided with an optical mirror and an optical lens, and emits the measurement light which is transmitted through the optical fiber 231, 336 in the direction approximately perpendicular to the axial direction (radial direction of the blood vessel). Also, the reflected light from the biological tissue with respect to the emitted measurement light is light-received and is transmitted to the steering control apparatus 103 side through the optical fiber 231, 336. Owing to the fact that the transmitting and receiving of such a light depending on the optical mirror and the optical lens is carried out during the radial operation of the imaging core 201, 301, it is possible to generate the line data for plural-generating the cross-sectional image inside the blood vessel in the axial direction.

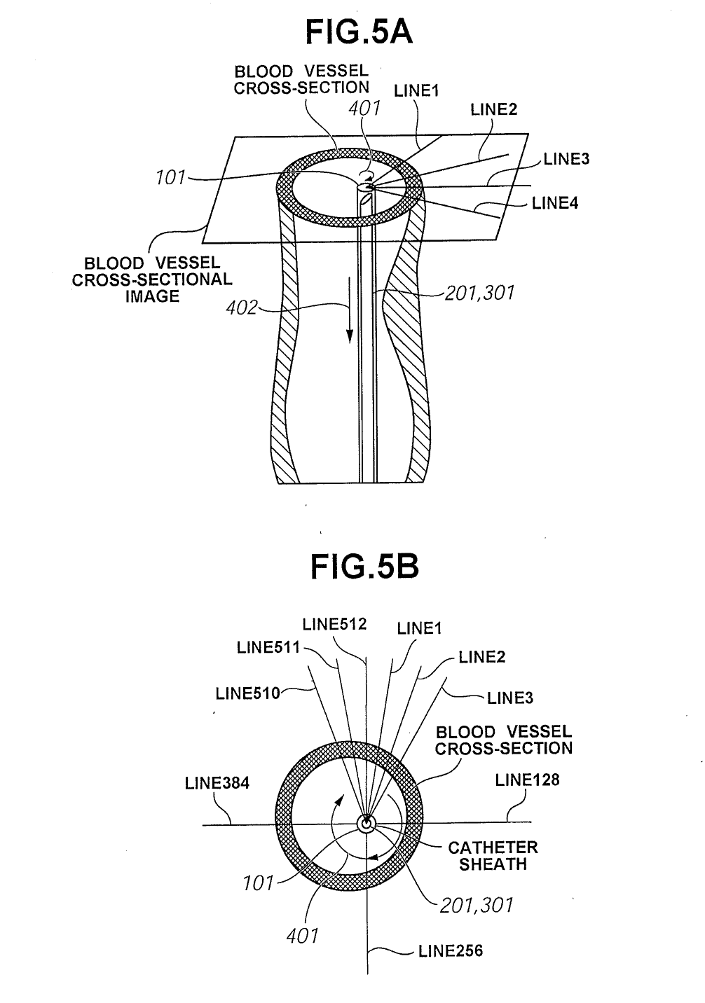

FIG. 5A is a diagram showing a state in which the measurement light is emitted toward the radial direction of the blood vessel while radially operating the imaging core 201, 301. As shown in FIG. 5A, because the measurement light is emitted while rotating the imaging core 201, 301 in the direction of the arrow 401, it is possible to light-receive the reflected light from the biological tissue at each position direction of the circumference of the transmitting and receiving unit (that is, line data at each position can be generated). “Line 1”, “Line 2”, . . . shown in FIG. 5A express the distribution of the data generated based on the reflected light from the biological tissue of the measurement light which is emitted with respect to the biological tissue at each position in the circumferential direction. In this manner, because the line data of a plurality of lines is generated at each position toward the circumferential direction in the axial direction, it is possible to generate the blood vessel cross-sectional image at each position in the axial direction.

FIG. 5B is a diagram showing one example of the number of times of the measurement-light emissions toward the radial direction of the blood vessel at a predetermined position in the axial direction. In the example of FIG. 5B, 512-times emissions are carried out while the imaging core 201, 301 is rotated once in the circumferential direction and the line data for 512 lines are generated.

5. Features and Operational Aspects of Signal Processing Unit

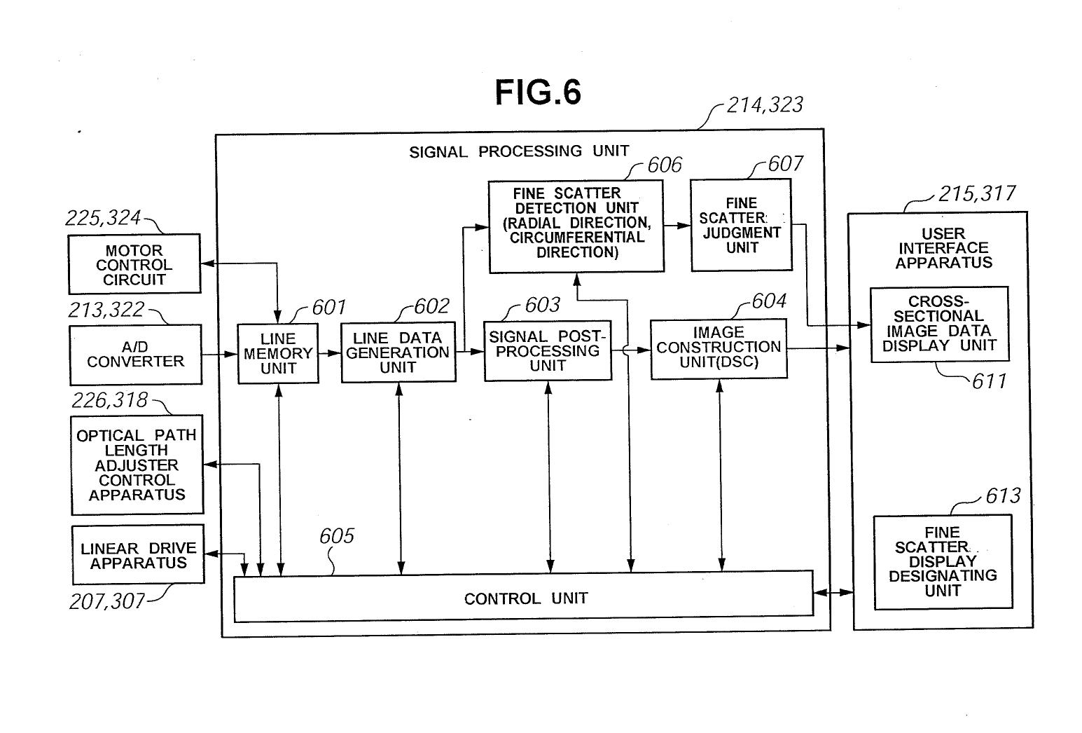

Referring to FIG. 6, set forth below is a discussion of the signal processing unit 214, 323 of the optical imaging diagnostic apparatus 100, including a description of operational aspects associated with the image developing process for developing or generating the blood vessel cross-sectional image and a fine scatter detection process for indicating the fine scatter in the blood vessel cross-sectional image. It is possible for the image developing process and the fine scatter detection process explained below to be realized by providing hardware for exclusive use or to be realized by a software (by executing a computer program) about the function of each portion.

FIG. 6 schematically illustrates features of the signal processing unit 214, 323 of the imaging diagnostic apparatus 100 used to perform the image developing process and fine scatter detection process. For purposes of simplifying the explanation, the description which follows focuses on the signal processing unit 214 of the optical coherent tomography apparatus 100 (in FIG. 2). Similar features and operational characteristics are used with an optical frequency domain imaging apparatus utilizing the wavelength sweep, and so a detailed discussion of the use in the context of an optical frequency domain imaging apparatus utilizing the wavelength sweep is not repeated here.

The coherent light data produced by the A/D converter 213 is processed in the line memory unit 601 such that the number of lines per one rotation of the radial scanning motor is 512 lines by using the signal of the encoder unit 206 of the radial scanning motor 205, which is outputted from the motor control circuit 225 and thereafter, it is outputted to the line data generation unit 602 in the succeeding stage.

In the line data generation unit 602, a line addition-averaging process, a filtering process, a logarithmic conversion process and the like are applied with respect to the coherent light data and the line data which is the coherent light intensity data in the depth direction of the biological tissue is generated and thereafter, the generated line data are outputted to the signal post-processing unit 603 in the succeeding stage. In the signal post-processing unit 603, a contrast adjustment, an intensity adjustment, a gamma correction, a frame correlation, a sharpness process and the like are carried out with respect to the line data and it is then outputted to the image construction unit (DSC) 604.

In the image construction unit 604, a blood vessel cross-sectional image is generated owing to the fact that the line data series of the polar coordinate are Rθ-converted and thereafter, the image is converted to a video signal and the blood vessel cross-sectional image is displayed on the cross-sectional image data display unit 611 of the user interface apparatus 215. In this embodiment disclosed by way of example, the blood vessel cross-sectional image is generated from 512 lines of data, but the apparatus and method here are not limited in that respect.

Also, the description above explains that the signal post-processing unit 603 directly processes the line data outputted from the line data generation unit 602, but the apparatus and method here are not limited in this regard as it is possible to include an arrangement in which the line data outputted from the line data generation unit 602 is stored in parallel in a storage unit as a file form in association with predetermined patient attribute information and measurement condition information. In this case, in the signal post-processing unit 603, the process described above is carried out in addition to a fact that the line data are read-out from the storage unit based on the instruction of a user. It is possible for the storage unit to be provided inside the control unit 605 or to be provided outside the signal processing unit 214 (for example, it is also possible for the DVD recorder 111-1 to function as the storage unit). Alternatively, it is also possible for the line data generation unit 602 itself to function as the storage unit.

The user interface apparatus 215 is provided with a fine (minute) scatter display instruction unit 613 and depending on this aspect, it is possible for a user to instruct so as to clearly indicate the existence of the fine scatter on the blood vessel cross-sectional image which is displayed on the cross-sectional image data display unit 611.

When an instruction by the fine scatter display instruction unit 613 is inputted, the instruction is transmitted to the fine scatter detection unit 606 through the control unit 605. In the fine scatter detection unit 606, a plurality of the line data used for the generation of the blood vessel cross-sectional image displayed on the cross-sectional image data display unit 611 is read-out from the storage unit and after carrying out a noise rejection process with respect to the plurality of line data, a differentiation process is carried out in the radial direction and in the circumferential direction (change of the signal intensity is calculated). Thus, for example, it is possible to detect the minute or fine scatter having a size of 20 μm or less. The fine scatter detection unit 606 is an example of a calculation means for respectively calculating signal-intensity change in a circumferential direction of a circumference of the transmitting and receiving unit and signal-intensity change in a radial direction of the body lumen with respect to a plurality of line data used for generating the displayed cross-sectional image.

A detection result in the fine scatter detection unit 606 is transmitted to a fine scatter judgment unit 607 where it is judged whether or not the fine scatter exists. In the fine scatter judgment unit 607, an intersection set of a detection result obtained by the differentiation process in the radial direction and a detection result obtained by the differentiation process in the circumferential direction is judged to be the fine scatter. However, it is also possible that a union set of a detection result obtained by the differentiation process in the radial direction and a detection result obtained by the differentiation process in the circumferential direction is judged to be the fine scatter. The fine scatter judgment unit 607 is an example of a specifying means for judging, based on a result calculated by the fine scatter detection unit 606, whether or not there exists scatter which scatters the light transmitted from the transmitting and receiving unit in a blood flow area inside the body lumen, and for specifying the position of the scatter on the displayed cross-sectional image when it is judged that scatter exists.

In the fine scatter judgment unit 607, the corresponding position on the cross-sectional image is calculated based on the position on the line data, which is judged to be the fine scatter and it is outputted to the cross-sectional image data display unit 611. Then, in the cross-sectional image data display unit 611, a predetermined marker is displayed at the calculated position on the cross-sectional image. The description below will describe the fine scatter detection process from the instruction input in the fine scatter display instruction unit 613 until the marker is displayed at the position of the fine scatter on the blood vessel cross-sectional image.

6. Fine Scatter Detection Process

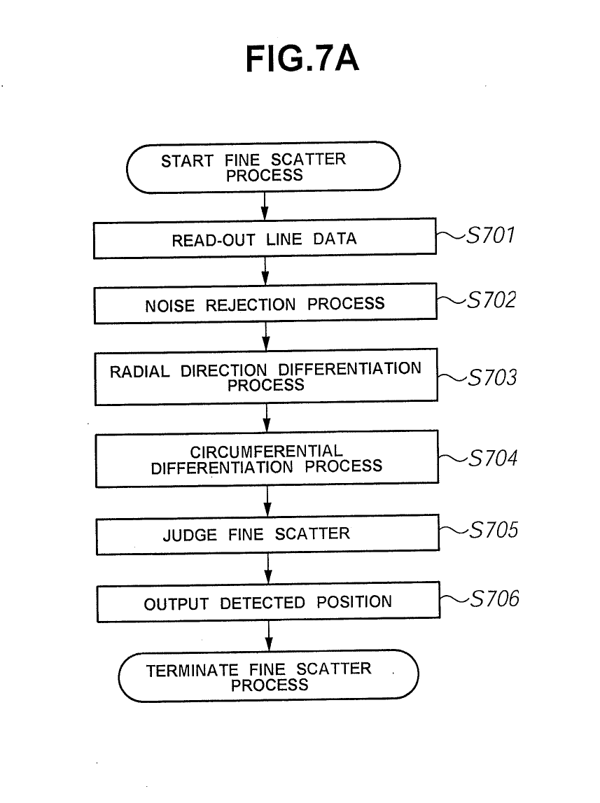

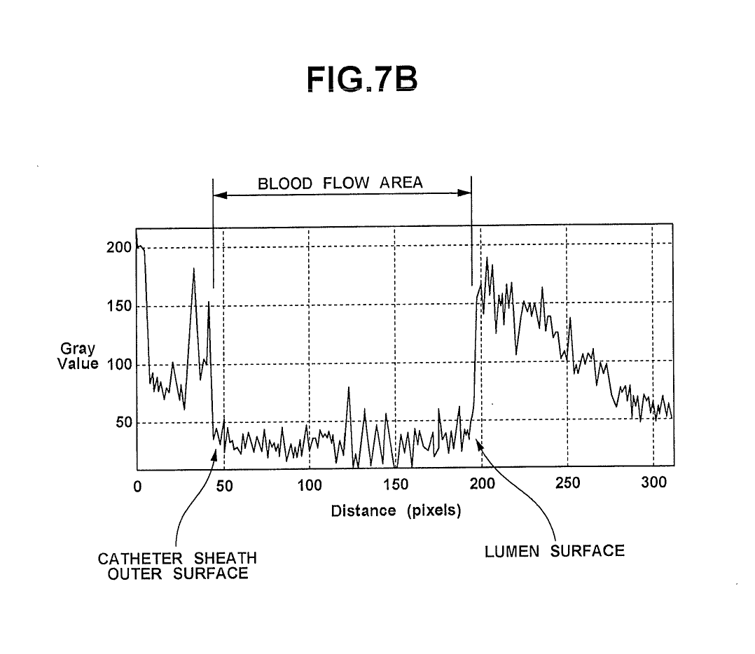

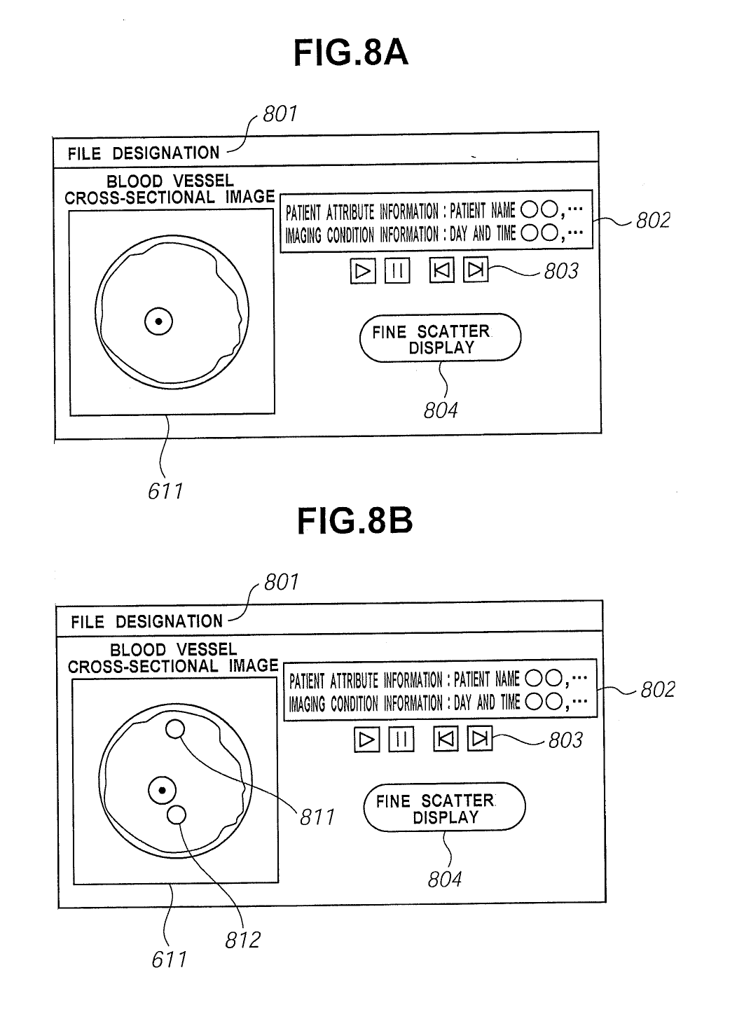

The fine scatter detection process will be explained below with reference to FIG. 7A, FIG. 7B, FIG. 8A and FIG. 8B. As shown in FIG. 8A, the user interface for carrying out the fine scatter detection process includes a file-designation button 801 for designating a desired file from a plurality of files in which a plurality of line data used for the generation of the blood vessel cross-sectional image are stored. The user interface also includes a cross-sectional image data display unit 611 which continuously displays the blood vessel cross-sectional image in real time at the time of measurement (or which continuously displays the blood vessel cross-sectional image based on a plurality of the line data stored in the designated file) and a display area 802 displaying various kinds of information (e.g., patient attribute information showing the attribute of a patient like the patient name, and measurement condition information like the measurement day and time, the settings at the time of measurement, and the like) with respect to the blood vessel cross-sectional image which is displayed on the cross-sectional image data display unit 611.

Further, in the cross-sectional image data display unit 611, there is provided an operation switch 803 for displaying a plurality of blood vessel cross-sectional images continuously, for temporarily-stopping the blood vessel cross-sectional image which is displayed continuously, for fast-forwarding or rewinding the display and the like.

With this construction, a user operates, for example, the file-designation button 801 and reads-out a desired file and concurrently, causes the blood vessel cross-sectional image of the desired position in the axial direction to be displayed on the cross-sectional image data display unit 611 by operating the operation switch 803. When the operation is completed by a user, the fine scatter detection process shown in FIG. 7A is started.

When the fine scatter detection process starts, in step S701, the fine scatter detection unit 606 reads-out the plurality of line data (line data for one blood vessel cross-sectional image, for example, 512 lines of line data) which are used to generate the blood vessel cross-sectional image displayed on the cross-sectional image data display unit 611 from the storage unit.

In step S702, a movement averaging process is carried out with respect to the plurality of line data read-out in step S701 (for example, filtering process is carried out by a five-point median filter) and the influence of the speckle noise is removed.

In step S703, the differentiation processes are carried out in the radial direction respectively with respect to all the plurality of line data in which the influence of the speckle noise is removed. By the differentiation processes, the fine scatter is detected. Specifically, the differentiation filter is applied to the respective line data, the fluctuation of the intensity value is calculated, and the fluctuation in which the calculated result reaches a predetermined threshold value or more is detected. This process is carried out with respect to all the plurality of line data. Thus, for example, it is possible to detect fine scatter of 20 μm or less from the respective line data.

Similarly, in step S704, the differentiation process is carried out in the circumferential direction respectively with respect to the plurality of line data in which the speckle noise is removed, and the fine scatter is detected (details of the differentiation process is similar as those in step S703). That is, the line data is differentiated.

With respect to the area which is applied with the differentiation filter in steps S703 and S704, the blood flow area inside the blood vessel (region from the outside of the outer surface of the catheter sheath of the optical probe unit 101 to the lumen position of the blood vessel, that is the area through which the physiological salt/saline solution flows in a case in which the blood is removed by flushing is a target (see one example of the blood flow area shown in FIG. 7B).

In step S705, the fine scatter judgment unit 607 judges the intersection of the detection results detected in step S703 and step S704 as the differential scatter. However, the apparatus and method here are not limited in this way as it is also possible to judge the union of the detection results detected in step S703 and step S704 as the differential scatter. The terms “intersection” and “union” as used here and in other parts of the description refer to those terms as used in mathematical set theory.

Step S706 involves calculating the position on the blood vessel cross-sectional image corresponding to the position on the line data which are judged as the differential scatter in step S705. Further, the calculated position of the blood vessel cross-sectional image is outputted to the cross-sectional image data display unit 611 and there is displayed, on the cross-sectional image data display unit 611, a predetermined marker at that position.

In FIG. 8B, reference numerals 811, 812 are one example of the marker which is displayed on the blood vessel cross-sectional image. It should be noted in the example of FIG. 8B that the position of the fine scatter is indicated clearly by a circle centering around the position on the blood vessel cross-sectional image of the fine scatter, but the style of the marker is not limited by this style and it is also possible to indicate the position clearly by another marker (e.g., arrow or the like).

In this manner, by clearly indicating the fine scatter whose existence is difficult to be confirmed on the displayed blood vessel cross-sectional image and which becomes a cause of the attenuation of the measurement light on the blood vessel cross-sectional image, it becomes possible for a user to distinguish the attenuation of the measurement light caused by the fine scatter and the attenuation of the measurement light by a cause other than the fine scatter.

It is understood from the explanation above that there is employed, in the optical imaging diagnostic apparatus relating to this embodiment disclosed by way of example, a construction in which it is possible for a user to instruct so as to clearly indicate the position of the fine scatter on the displayed blood vessel cross-sectional image.

Also, in case of obtaining an instruction from a user, there is employed a construction in which the fine scatter is detected by reading-out the line data used for the generation of the blood vessel cross-sectional image and by applying the differentiation process thereon in the radial direction and in the circumferential direction. Further, there is employed a construction in which the position on the blood vessel cross-sectional image of the detected fine scatter is calculated and the marker is displayed at that position.

Thus, it is possible for a user to identify the presence or non-presence of the fine scatter on the blood vessel cross-sectional image.

### Second Embodiment

In the first embodiment described above as an example of the apparatus and method disclosed here, when the fine scatter is detected, the position of the fine scatter on the blood vessel cross-sectional image is calculated and the marker is displayed at that position, but the apparatus and method are not limited in this way. For example, it is also possible to employ a construction in which the blood vessel cross-sectional image is corrected so as to display a backward area of the fine scatter (that is, the area in which the measurement light is attenuated by the fine scatter) of that position that is emphasized. In the description below describing such an alternative embodiment, the description focuses primarily upon the difference between this second embodiment relative to the first embodiment described above.

1. Features and Operational Aspects of Signal Processing Unit

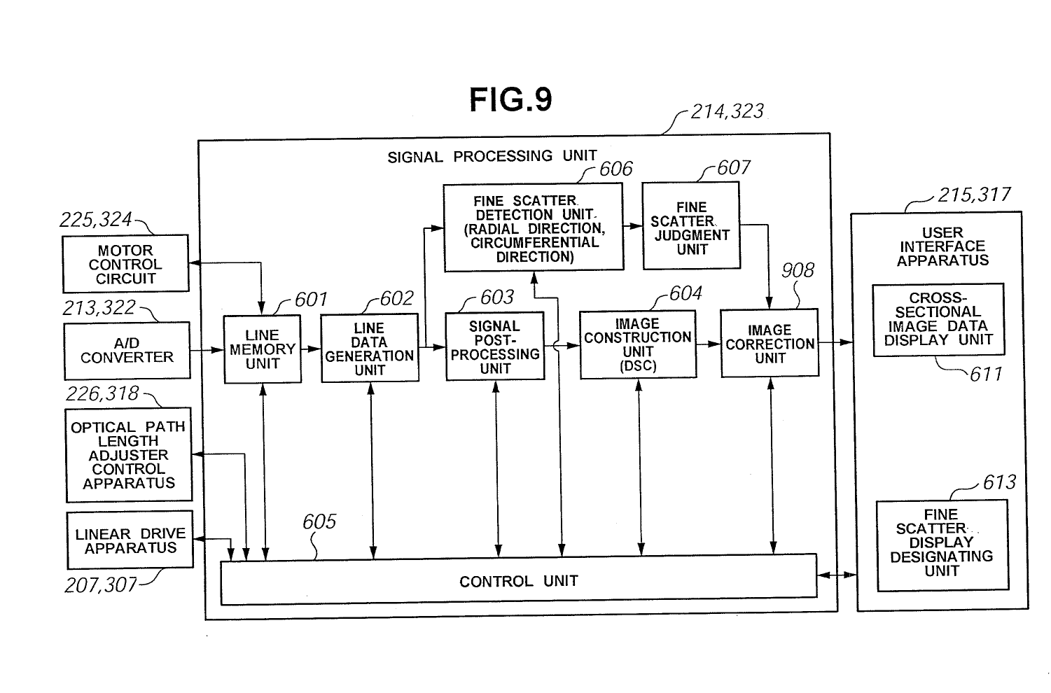

Features and operational aspects of the signal processing unit associated with this second embodiment are discussed below with reference to FIG. 9. In FIG. 9, features similar to those described above are identified by common reference numerals, and a detailed description of those common features is not repeated here.

In FIG. 9, the fine scatter judgment unit 607 calculates the position on the blood vessel cross-sectional image based on the position on the line data which are judged as the fine scatter, and concurrently the area on the cross-sectional image which corresponds to the backward area in the radial direction of that position is calculated. Further, with respect to the calculated backward area on the cross-sectional image, transmission to an image correction unit 908 is carried out.

In the image correction unit 908, each pixel on the cross-sectional image in the backward area is corrected, and the backward area on the cross-sectional image is displayed in a manner in which it is emphasized. The image correction unit 908 is an example of a correction means for correcting the displayed cross-sectional image of a backward area which is positioned backward in the radial direction of the body lumen.

2. Fine Scatter Detection Process

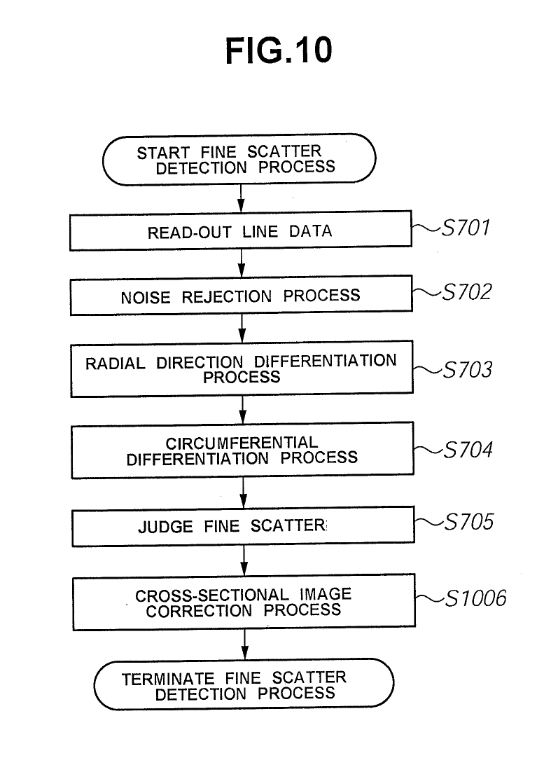

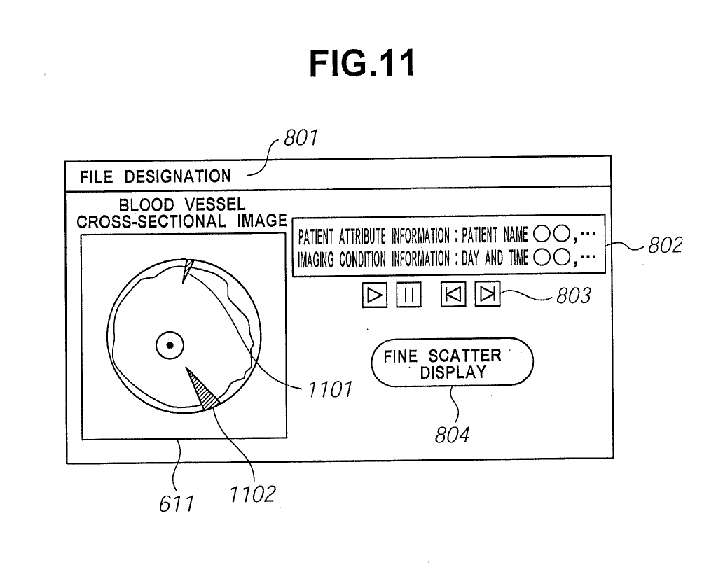

Next, the fine scatter detection process according to this embodiment described by way of example will be described with reference to FIG. 10 and FIG. 11. FIG. 10 illustrates aspects of the fine scatter detection process for detecting a fine scatter in the displayed blood vessel cross-sectional image and for displaying a backward area of the detected fine scatter in an emphasized manner. FIG. 11 shows one example of the user interface for carrying out the fine scatter detection process with respect to the displayed blood vessel cross-sectional image.

The processes in steps S701 to S705 are the same as the processes in steps S701 to S705 explained in FIG. 7A, and so a detailed description of such processes is not repeated here.

In step S1006, the fine scatter judgment unit 607 specifies the backward area on the blood vessel cross-sectional image of the fine scatter which is detected. Further, the image correction unit 908 corrects the blood vessel cross-sectional image with respect to the specified backward area. That is, the image correction unit 908 corrects the displayed cross-sectional image of the backward area to display each pixel of the backward area in the displayed cross-sectional image in an emphasized or highlighted manner to distinguish the pixels of the backward area in a visually distinguishing manner relative to the portion of the displayed cross-sectional image adjacent the backward area.

FIG. 11 is a diagram showing one example of the blood vessel cross-sectional image after carrying out the cross-sectional image correction process in step S1006. In FIG. 11, reference numerals 1101 and 1102 indicate areas which are displayed in an emphasized or highlighted manner in the backward area of the fine scatter which is detected.

In this manner, by employing an arrangement in which the backward area of the fine scatter is displayed by being emphasized, it becomes possible to make a user recognize on the blood vessel cross-sectional image that the aforementioned area is an area which received the influence of the attenuation of the measurement light caused by the fine scatter.

### Third Embodiment

The second embodiment described above employs a construction involving displaying the backward area of the detected fine scatter in an emphasized manner on the blood vessel cross-sectional image, and so a user recognizes that the aforementioned area is an area which received the influence of the attenuation of the measurement light caused by the fine scatter. But the apparatus and method here are not limited in this regard.

For example, with respect to the backward area which receives the influence of the attenuation of the measurement light caused by the fine scatter, it is also possible to employ an arrangement in which the blood vessel cross-sectional image is corrected to remove the influence of the attenuation of the measurement light. Hereinafter, such an embodiment will be explained.

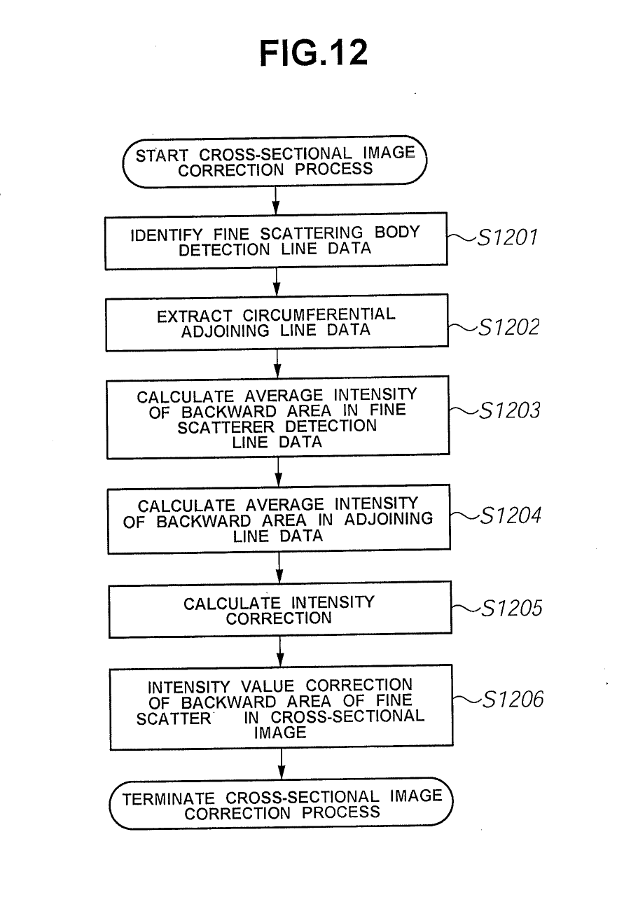

FIG. 12 is a flowchart showing operational aspects of the cross-sectional image correction process (step S1006 of FIG. 10) in this embodiment.

In step S1201, the fine scatter judgment unit 607 identifies the line data (fine scatter detection line data) in which the fine scatter is detected in step S706.

In step S1202, there are extracted the line data (adjacent line data) which are adjacent to the fine scatter detection line data identified in step S1201 in a circumferential direction (there are extracted line data which are adjacent with the fine scatter detection line data in a circumferential direction on the blood vessel cross-sectional image).

In step S1203, there is calculated an average intensity in the backward area of the fine scatter of the fine scatter detection line data identified in step S1201. Also, in step S1204, there is calculated an average intensity in the backward area of the adjacent line data extracted in step S1202.

In step S1205, by comparing the average intensity calculated in step S1203 and the average intensity calculated in step S1204, an intensity correction value in the backward area of the fine scatter of the fine scatter detection line data is calculated.

In step S1206, based on the intensity correction value calculated in step S1205, the image correction unit 908 corrects the intensity value of each pixel of the backward area of the fine scatter in the blood vessel cross-sectional image.

As is clear from the explanation above, according to this embodiment, it is possible, with respect to the backward area which receives the influence of the attenuation of the measurement light caused by the fine scatter, to remove the influence of the attenuation of the measurement light.

### Fourth Embodiment

The third embodiment described above employs a construction in which the intensity correction value in the backward area of the fine scatter detection line data is calculated by using the adjacent line data, but the apparatus and method here are not limited in this regard. For example, the degree of influence of the attenuation of the measurement light caused by the fine scatter differs in response to the size of the fine scatter, so that it is also possible to employ a construction in which the attenuation curve is preliminarily kept or held for every size of the fine scatter and the intensity correction value is calculated by using the attenuation curve based on the detected size of the fine scatter.

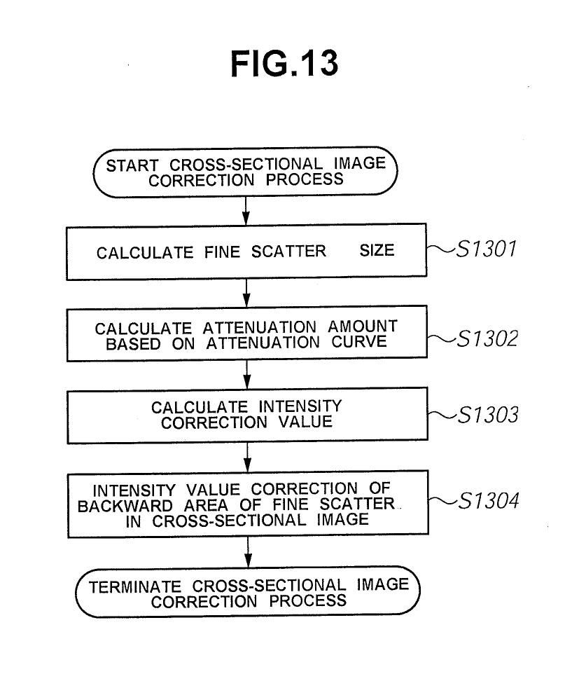

FIG. 13 illustrates operational aspects of the cross-sectional image correction process (step S1006 of FIG. 10) in this embodiment.

In step S1301, the fine scatter judgment unit 607 calculates the size of the fine scatter which is detected in step S706. In step S1302, there is extracted the attenuation curve corresponding to the size of the fine scatter which is calculated in step S1301 (attenuation curve corresponding to the size of the fine scatter, which is calculated, in step S1301, from within the attenuation curves kept or held preliminarily for every size of the fine scatter) and the amount of attenuation at each position in the radial direction is calculated based on that extracted attenuation curve. The control unit 605 which carries out the routine shown in FIG. 13, including steps S1302, is an example of a holding means for preliminarily holding the attenuation curve indicating light attenuation caused by scatter for every size of the scatter.

In step S1303, the intensity correction value is calculated based on the amount of attenuation which is calculated in step S1302.

In step S1304, the image correction unit 908 calculates the intensity value of each pixel of the backward area of the fine scatter in the blood vessel cross-sectional image based on the intensity correction value which is calculated in step S1303.

The explanation above points out that this embodiment makes it possible to remove the influence of the attenuation of the measurement light with respect to the backward area which receives the influence of the attenuation of the measurement light caused by the fine scatter.

The detailed description above describes features and aspects of embodiments of an optical imaging diagnostic apparatus and a display control method. The invention is not limited, however, to the precise embodiments and variations described. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. An optical imaging diagnostic apparatus in which a probe including a transmitting and receiving unit for carrying out continuous transmitting of light which is reflected from biological tissue and received as obtained reflected light by the transmitting and receiving unit, with the transmitting and receiving unit being axially movable inside a body lumen while also rotating the transmitting and receiving unit, to generate a plurality of cross-sectional images in an axial direction of the biological tissue using line data of coherent light produced by interference between the obtained reflected light and reference light, with the cross-sectional images being displayed, the apparatus comprising:
calculation means for respectively calculating signal-intensity change in a circumferential direction of a circumference of the transmitting and receiving unit and signal-intensity change in a radial direction of the body lumen with respect to a plurality of line data used for generating the displayed cross-sectional image;
specifying means for judging, based on a result calculated by the calculation means, whether or not there exists scatter which scatters the light transmitted from the transmitting and receiving unit in a blood flow area inside the body lumen, and for specifying a position of the scatter on the displayed cross-sectional image when it is judged that the scatter exists; and
a display which displays on the display a marker indicating that the scatter exists and indicating on the displayed cross-sectional image the position of the scatter specified by the specifying means.

2. The optical imaging diagnostic apparatus according to claim 1, wherein the specifying means judges that the scatter exists at a position which is a union or an intersection of a position at which signal-intensity change in the circumferential direction of the circumference of the transmitting and receiving unit reaches a predetermined value or more and a position at which signal-intensity change in the radial direction of the body lumen reaches a predetermined value or more.

3. The optical imaging diagnostic apparatus according to claim 1, further comprising, with respect to the specified position, correction means for correcting the displayed cross-sectional image of a backward area which is positioned backward in the radial direction of the body lumen.

4. The optical imaging diagnostic apparatus according to claim 3, wherein the correction means corrects the displayed cross-sectional image of the backward area to display each pixel of the backward area in the displayed cross-sectional image in an emphasized manner to distinguish the pixels of the backward area in a visually distinguishing manner relative to a portion of the displayed cross-sectional image adjacent the backward area.

5. The optical imaging diagnostic apparatus according to claim 3, wherein the correction unit calculates a correction value by comparing signal intensity of the line data in which it is judged that the scatter exists and signal intensity of line data circumferentially neighboring the line data in which it is judged that the scatter exists, and corrects the intensity value of each pixel of the backward area in the displayed cross-sectional image using the calculated correction value.

6. The optical imaging diagnostic apparatus according to claim 3, further comprising:
holding means for preliminarily holding an attenuation curve indicating light attenuation caused by scatter for every size of the scatter, wherein
the correction unit calculates a correction value of the backward area in the displayed cross-sectional image based on the attenuation curve by calculating the size of the scatter judged in the specifying unit, and corrects the intensity value of each pixel of the backward area in the displayed cross-sectional image depending on the calculated correction value.

7. A display control method in an optical imaging diagnostic apparatus, which apparatus comprises a probe including a transmitting and receiving unit for carrying out continuous transmitting of light which is reflected from biological tissue and received as obtained reflected light by the transmitting and receiving unit, with the transmitting and receiving unit being axially movable inside a body lumen while also rotating, to generate a plurality of cross-sectional images in an axial direction of the biological tissue using line data of coherent light produced by interference between the obtained reflected light and reference light, with the cross-sectional images being displayed, the method comprising:
calculating both signal-intensity change in a circumferential direction of a circumference of the transmitting and receiving unit and signal-intensity change in a radial direction of the body lumen respectively with respect to plural line data used to generate the displayed cross-sectional image;
judging, based on both the signal-intensity change in the circumferential direction of the circumference of the transmitting and receiving unit and the signal-intensity change in the radial direction of the body lumen, whether or not scatter exists which scatters the light transmitted from the transmitting and receiving unit in a blood flow area of the body lumen;
specifying, when it is determined that scatter exist, a position of the scatter on the displayed cross-sectional image; and
the specifying of the position of the scatter on the displayed cross-sectional image including displaying a marker on the displayed cross-sectional image which identifies the position of the scatter.

8. The display control method according to claim 7, wherein the judging of whether or not the scatter exists comprises judging that scatter exists at a union or an intersection of a position at which signal-intensity change in the circumferential direction of the circumference of the transmitting and receiving unit reaches a predetermined value or more and a position at which signal-intensity change in the radial direction of the body lumen reaches a predetermined value or more.

9. The display control method according to claim 7, further comprising correcting the displayed cross-sectional image of a backward area which is positioned backward in the radial direction of the body lumen.

10. The display control method according to claim 9, wherein the correcting of the displayed cross-sectional image of the backward area comprises correcting the displayed cross-sectional image of the backward area to display each pixel of the backward area in the displayed cross-sectional image in a highlighted manner to distinguish the pixels of the backward area in a visually distinguishing manner relative to a portion of the displayed cross-sectional image adjacent the backward area.

11. The display control method according to claim 9, wherein the correcting of the displayed cross-sectional image of the backward area comprises comparing signal intensity of the line data in which it is judged that the scatter exists and signal intensity of line data circumferentially neighboring the line data in which it is judged that the scatter exists, and correcting the intensity value of each pixel of the backward area in the displayed cross-sectional image using the calculated correction value.

12. The display control method according to claim 9, further comprising preliminarily holding an attenuation curve indicating light attenuation caused by scatter for every size of the scatter, and the correcting of the displayed cross-sectional image of the backward area comprises calculating a correction value of the backward area in the displayed cross-sectional image based on the attenuation curve by calculating the size of the scatter judged in the specifying unit, and corrects the intensity value of each pixel of the backward area in the displayed cross-sectional image depending on the calculated correction value.
